(19) United States
(12) Patent Application Publication
(10) Pub. No.: US 2016/0051361 A1
(43) Pub. Date: Feb. 25, 2016

(54) Accommodating Intraocular Lens

(71) Applicant: EMMETROPE INCORPORATED, La Canada, CA (US)

(72) Inventor: Andrew F. Phillips, La Canada, CA (US)

(73) Assignee: EMMETROPE INCORPORATED, La Canada, CA (US)

(21) Appl. No.: 14/464,975

(22) Filed: Aug. 21, 2014

(51) Int. Cl. A61F 2/16

(52) U.S. Cl. CPC A61F 2/1629; A61F 2002/1681; A61F 2220/0091

(57) ABSTRACT

An intraocular lens (IOL) includes a first optic, a first set of haptics extending from the first optic, a second optic, a second set of haptics extending from the second optic, and a hinge joining the first and second sets of haptics. The IOL is subject to a pre-bias that biases the first and second optics away from one another along an anterior-posterior (A-P) axis. The IOL is also provided with a restraining element that restrains the optics relative to each other in a stressed, planar, non-accommodating configuration during implantation and a post-operative period. The restraining element extends across a portion of at least one of the first and second optics, but is not reliant on use of the haptics for support thereof. Upon release of the restraining element, the optics can move relative to each other along the A-P axis in accommodation.

### BACKGROUND OF THE INVENTION

1. Field of the Invention

This invention relates broadly to ophthalmic implants. More particularly, this invention relates to intraocular lenses which are focusable and allow for accommodation for near vision.

2. State of the Art

Referring to FIG. 1, the human eye 10 generally comprises a cornea 12, an iris 14, a ciliary body (muscle) 16, a capsular bag 18 having an anterior wall 20 and a posterior wall 22, and a natural crystalline lens 24 contained with the walls of the capsular bag. The capsular bag 18 is connected to the ciliary body 16 by means of a plurality of zonules 26 which are strands or fibers. The ciliary body 16 surrounds the capsular bag 18 and lens 24, defining an open space, the diameter of which depends upon the state (relaxed or contracted) of the ciliary body 16.

When the ciliary body 16 relaxes, the diameter of the opening increases, and the zonules 26 are pulled taut and exert a tensile force on the anterior and posterior walls 20, 22 of the capsular bag 18, tending to flatten it. As a consequence, the lens 24 is also flattened, thereby undergoing a decrease in focusing power. This is the condition for normal distance viewing. Thus, the emmetropic human eye is naturally focused on distant objects.

Through a process termed accommodation, the human eye can increase its focusing power and bring into focus objects at near. Accommodation is enabled by a change in shape of the lens 24. More particularly, when the ciliary body 16 contracts, the diameter of the opening is decreased thereby causing a compensatory relaxation of the zonules 26. This in turn removes or decreases the tension on the capsular bag 18, and allows the lens 24 to assume a more rounded or spherical shape. This rounded shape increases the focal power of the lens such that the lens focuses on objects at near.

As such, the process of accommodation is made more efficient by the interplay between stresses in the ciliary body and the lens. When the ciliary body relaxes and reduces its internal stress, there is a compensatory transfer of this stress into the body of the lens, which is then stretched away from its globular relaxed state into a more stressed elongated conformation for distance viewing. The opposite happens as accommodation occurs for near vision, where the stress is transferred from the elongated lens into the contracted ciliary body.

In this sense, referring to FIG. 2, there is conservation of potential energy (as measured by the stress or level of excitation) between the ciliary body and the crystalline lens from the point of complete ciliary body relaxation for distance vision through a continuum of states leading to full accommodation of the lens.

As humans age, there is a general loss of ability to accommodate, termed “presbyopia”, which eventually leaves the eye unable to focus on near objects. In addition, when cataract surgery is performed and the natural crystalline lens is replaced by an artificial intraocular lens, there is generally a complete loss of the ability to accommodate. This occurs because the active muscular process of accommodation involving the ciliary body is not translated into a change in focusing power of the implanted artificial intraocular lens.

There have been numerous attempts to achieve at least some useful degree of accommodation with an implanted intraocular lens which, for various reasons, fall short of being satisfactory. In U.S. Pat. No. 4,666,446 to Koziol et al., there is shown an intraocular lens having a complex shape for achieving a bi-focal result. The lens is held in place within the eye by haptics which are attached to the ciliary body. However, the implant requires the patient to wear spectacles for proper functioning. Another device shown in U.S. Pat. No. 4,994,082 to Richards et al., also utilizes a lens having regions of different focus, or a pair of compound lenses, which are held in place by haptics attached to the ciliary body. In this arrangement, contraction and relaxation of the ciliary muscle causes the haptics to move the lens or lenses, thereby altering the effective focal length. There are numerous other patented arrangements which utilize haptics connected to the ciliary body, or are otherwise coupled thereto, such as are shown in U.S. Pat. Nos. 4,932,966 to Christie et al., U.S. Pat. No. 4,888,012 to Horne et al. and U.S. Pat. No. 4,892,543 to Turley, and rely upon the ciliary muscle to achieve the desired alteration in lens focus.

In any arrangement that is connected to the ciliary body, by haptic connection or otherwise, extensive erosion, scarring, and distortion of the ciliary body usually results. Such scarring and distortion leads to a disruption of the local architecture of the ciliary body and thus causes failure of the small forces to be transmitted to the intraocular lens. Thus, for a successful long-term implant, connection and fixation to the ciliary body is to be avoided if at all possible.

In U.S. Pat. No. 4,842,601 to Smith, there is shown an accommodating intraocular lens that is implanted into and floats within the capsular bag. The lens comprises front and rear flexible walls joined at their edges, which bear against the anterior and posterior inner surfaces of the capsular bag. Thus, when the zonules exert a tensional pull on the circumference of the capsular bag, the bag, and hence the intraocular lens, is flattened, thereby changing the effective power of refraction of the lens. The implantation procedure requires that the capsular bag be intact and undamaged and that the lens itself be dimensioned to remain in place within the bag without attachment thereto. Additionally, the lens must be assembled within the capsular bag and biasing means for imparting an initial shape to the lens must be activated within the capsular bag. Such an implantation is technically quite difficult and risks damaging the capsular bag, inasmuch as most of the operations involved take place with tools which invade the bag. In addition, the Smith arrangement relies upon pressure from the anterior and posterior walls of the capsular bag to deform the lens, which requires that the lens be extremely resilient and deformable. However, the more resilient and soft the lens elements, the more difficult assembly within the capsular bag becomes. Furthermore, fibrosis and stiffening of the capsular remnants following cataract surgery may make this approach problematic.

U.S. Pat. No. 6,197,059 to Cumming and U.S. Pat. No. 6,231,603 to Lang each disclose an intraocular lens design where the configuration of a hinged lens support ostensibly allows the intraocular lens to change axial position in response to accommodation and thus change effective optical power. U.S. Pat. No. 6,299,641 to Woods describes another intraocular lens that also increases effective focusing power as a result of a change in axial position during accommodation. In each of these intraocular lenses, a shift in axial position and an increase in distance from the retina results in a relative increase in focusing power. All lenses that depend upon a shift in the axial position of the lens to achieve some degree of accommodation are limited by the amount of excursion possible during accommodation.

U.S. Pat. No. 5,607,472 to Thompson describes a dual-lens design. Prior to implantation, the lens is stressed into a non-accommodative state with a gel forced into a circumferential expansion channel about the lens. At implantation, the surgeon must create a substantially perfectly round capsullorrhexis, and insert the lens therethrough. A ledge adjacent to the anterior flexible lens is then bonded 360° around (at the opening of the capsulorrhexis) by the surgeon to the anterior capsule to secure the lens in place. This approach has numerous drawbacks, a few of which follow. First, several aspects of the procedure are substantially difficult and not within the technical skill level of many eye surgeons. For example, creation of the desired round capsullorrhexis within the stated tolerance required is particularly difficult. Second, the bonding “ledge” may disrupt the optical image produced by the adjacent optic. Third, intraocular bonding requires a high degree of skill, and may fail if the capsullorrhexis is not 360° round. Fourth, the proposed method invites cautionary speculation as to the result should the glue fail to hold the lens in position in entirety or over a sectional region. Fifth, it is well known that after lens implantation surgery the capsular bag, upon healing, shrinks Such shrinking can distort a lens glued to the bag in a pre-shrunk state, especially since the lens is permanently affixed to a structure which is not yet in equilibrium. Sixth, Thompson fails to provide a teaching as to how or when to release the gel from the expansion channel; i.e., remove the stress from the lens. If the gel is not removed, the lens will not accommodate. If the gel is removed during the procedure, the lens is only in a rounded non-stressed shape during adhesion to the capsule, and it is believed that the lens will fail to interact with the ciliary body as required to provide the desired accommodation as the capsular bag may change shape in the post-operative period. If the gel is removed after the procedure, it is ostensibly via an additional invasive surgical procedure. In view of these problems, it is doubtful that the lens system disclosed by Thompson can be successfully employed.

Co-owned U.S. Pat. No. 7,601,169 to Phillips describes an intraocular lens for placement within the capsular bag. The lens includes an optic portion and a surrounding peripheral portion. A bias element is provided to anteriorly vault the optic portion relative to the peripheral portion. A restraint is provided to counter the bias element, and constrain the lens in stressed relatively planar configuration during surgical implantation and a healing period during which the eye is maintained under cycloplegia and the peripheral portion and capsular bag are permitted to naturally fuse together. Then, post-healing, the restraint is removed permitting the bias element to vault the optic portion anteriorly into a non-stressed state such that the optic portion is at an increased distance from the retina relative to the stressed state and has a resulting increased optical power, and wherein the optical power of the lens is adjustable in response to stresses induced by the eye. The Phillips system uses only a single optic.

### SUMMARY OF THE INVENTION

An intraocular lens (IOL) according to the invention includes two optics that are adapted to move along an anterior-posterior axis on a haptic system to provide an accommodative effect. The IOL includes a posterior first optic, a first set of haptics extending from the periphery of the first optic for stabilizing the first optic within the lens capsule and/or at the ciliary body, an anterior second optic, a second set of haptics extending from the periphery of the second optic for stabilizing the second optic within the lens capsule and/or at the ciliary body, and a hinge joining the first and second sets of haptics.

For the lens to function optimally in accommodation, at least one of the first and second set of haptics are subject to a pre-bias such that the haptics are naturally biased or otherwise urged to rotate or bend at their respective optic-haptic junctions to move one optic away from the other optic along the anterior-posterior axis. Such pre-bias is preferably applied by integration of a bias element at the haptic-optic junction, at the hinge between the first and second sets of haptics, or a combination thereof. Such bias element may comprise a resilient polymer and may be integrated in the construction of the optic-haptic junction and/or the hinge.

In accord with another aspect of the lens, the lens is restrained against the bias in a stressed state and within a more planar configuration, with the first optic retained at a first location or within a first distance of the second optic. A restraining element is provided to the lens for temporarily restraining the lens in the stressed, planar, non-accommodating configuration during implantation and a post-operative period. The restraining element comprise an element of material extending across a portion of at least one of the first and second optics within 5 mm of the optical center, and more preferably within 4 mm of the optical center. The restraining element preferably extends diametrically across the optic without support of the haptics. The restraining element preferably extends in a direction transverse to a long axis that is defined across an optic and the set of haptics extending therefrom. In accord with embodiments, the restraining element extends orthogonal to the long axis. In accord with embodiments, the restraining element extends parallel to a short axis orthogonal to the long axis. In accord with an embodiment, the restraining element extends coaxial with the short axis. In accord with an embodiment, a plurality of restraining elements extends parallel to the short axis. In accord with an embodiment, at least one restraining element extends at an angle relative to each of the long and short axes.

More particularly, at least one of the optics preferably includes peripheral holes or peripheral mounts at which the restraining element can be attached. In accord with an embodiment, the restraining element is coupled to a periphery of the first optic, extends over the second optic, and then is coupled back to another peripheral location of the first optic that is rotationally offset from the haptics. The restraining element is of such a length or otherwise dimensioned so as to retain the first and second optics in a defined spaced relationship. The restraining element may be a suture material.

The restraining element is preferably constructed of a material that is laser-releasable or chemically-releasable, or bioabsorbable material, such that the element automatically releases the retained configuration of the two optics relative to each other after a post-operative period, or may be released under the control of an eye surgeon, preferably via a non-surgically invasive means such as via a laser or a chemical agent added to the eye.

Generally, the method for implanting the intraocular lens includes (a) inducing cycloplegia; (b) providing the intraocular lens having a first optic portion, a first set of haptics extending from the first optic portion, a second optic portion, a second set of haptics extending from the second optic portion, a flexible hinge at the ends of the first and second sets of haptics, and an as manufactured inherent bias induced between at least one of (i) the first optic portion and the first set of haptics, (ii) the second optic portion and the second set of haptics, and (iii) and the hinge, the intraocular lens being held in a stressed, planar, non-accommodating state by a restraining element that extends across the lens in a direction that is transverse to the long axis of the intraocular lens such that the intraocular lens has a lower optical power relative to an accommodating non-stressed state of the lens; (c) inserting the stressed state intraocular lens into a capsular bag of the eye; (d) maintaining cycloplegia until the capsular bag physiologically affixes to the intraocular lens; and (e) releasing the restraining means to permit the first and second optics of the intraocular lens to move relative to each other along the anterior-posterior axis from the stressed state into the non-stressed state into a configuration in which the intraocular lens has an increased optical power, and wherein the optical power of the intraocular lens is reversibly adjustable in response to stresses induced by the eye such that the lens can accommodate.

More particularly, according to a preferred method of implantation, the ciliary body muscle is pharmacologically induced into a relaxed stated (cycloplegia), a capsulorrhexis is performed on the lens capsule, and the natural lens is removed from the capsule. The prosthetic lens is then placed within the lens capsule. According to a preferred aspect of the invention, the ciliary body is maintained in the relaxed state for the duration of the time required for the capsule to naturally heal and shrink about the lens; i.e., possibly for several weeks. After healing has occurred, the restraining element automatically or under surgeon control releases the lens from the stressed state. The ciliary body and lens may then interact in a manner substantially similar to the physiological interaction between the ciliary body and a healthy natural crystalline lens to allow the optics to move relative to each other.

Alternatively, a fully relaxed lens (i.e., without restraining element) can be coupled to a fully stressed and contracted ciliary body.

The intraocular lens of the invention is compatible with modern cataract surgery techniques. The lens utilizes an axial displacement of one optic relative to another optic to achieve a large increase in optical power of the implanted lens.

Additional objects and advantages of the invention will become apparent to those skilled in the art upon reference to the detailed description taken in conjunction with the provided figures.

### BRIEF DESCRIPTION OF THE DRAWINGS

Prior Art FIG. 1 is a diagrammatic view of a cross-section of a normal eye.

Prior Art FIG. 2 is a graph of the stresses on the ciliary body-crystalline lens system of the eye in a continuum of states between distance vision and full accommodation.

FIG. 3 is a schematic plan view of an intraocular lens according to a first embodiment of the invention in restrained and stressed configuration.

FIG. 4 is a schematic side view of the intraocular lens of FIG. 3 in the restrained and stressed configuration.

FIG. 5 is a schematic side view of the intraocular lens of FIG. 4 in the released and unconstrained configuration.

FIG. 6 is a schematic plan view of an intraocular lens according to a second embodiment of the invention in restrained and stressed configuration.

FIG. 7 is a schematic side view of the intraocular lens of FIG. 6 in the restrained and stressed configuration.

FIG. 8 is a schematic side view of the intraocular lens of FIG. 7 in the released and unconstrained configuration.

FIG. 9 is a schematic plan view of an intraocular lens according to a third embodiment of the invention in restrained and stressed configuration.

FIG. 10 is a schematic side view of the intraocular lens of FIG. 9 in the restrained and stressed configuration.

FIG. 11 is a schematic side view of the intraocular lens of FIG. 10 in the released and unconstrained configuration.

FIG. 12 is a schematic side view of another embodiment of an intraocular lens in a restrained and stressed configuration.

FIG. 13 is a schematic side view of another embodiment of an intraocular lens in a restrained and stressed configuration.

FIG. 14 is a schematic side view of another embodiment of an intraocular lens in a restrained and stressed configuration.

### DETAILED DESCRIPTION OF THE PREFERRED EMBODIMENTS

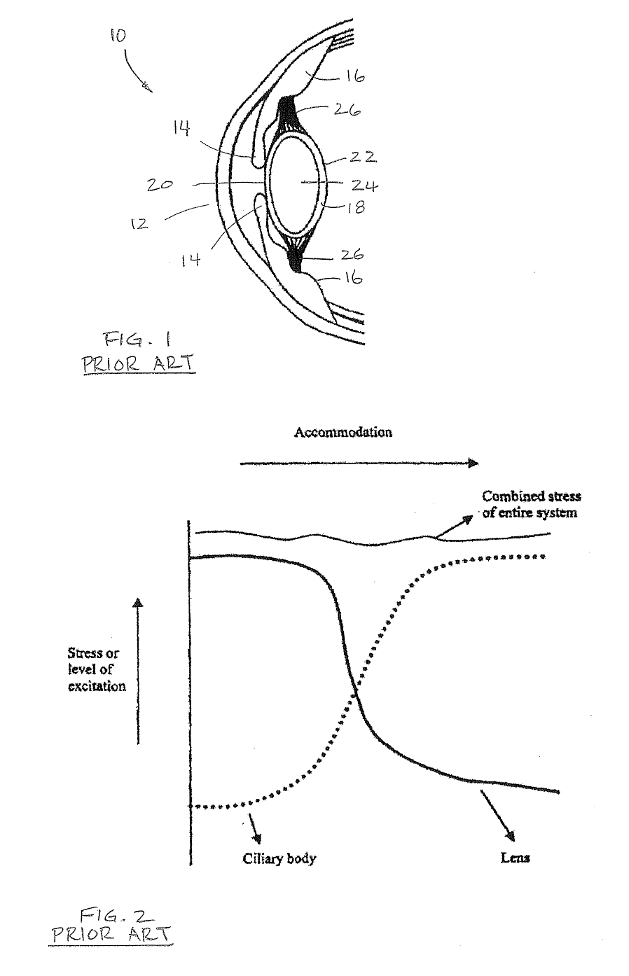

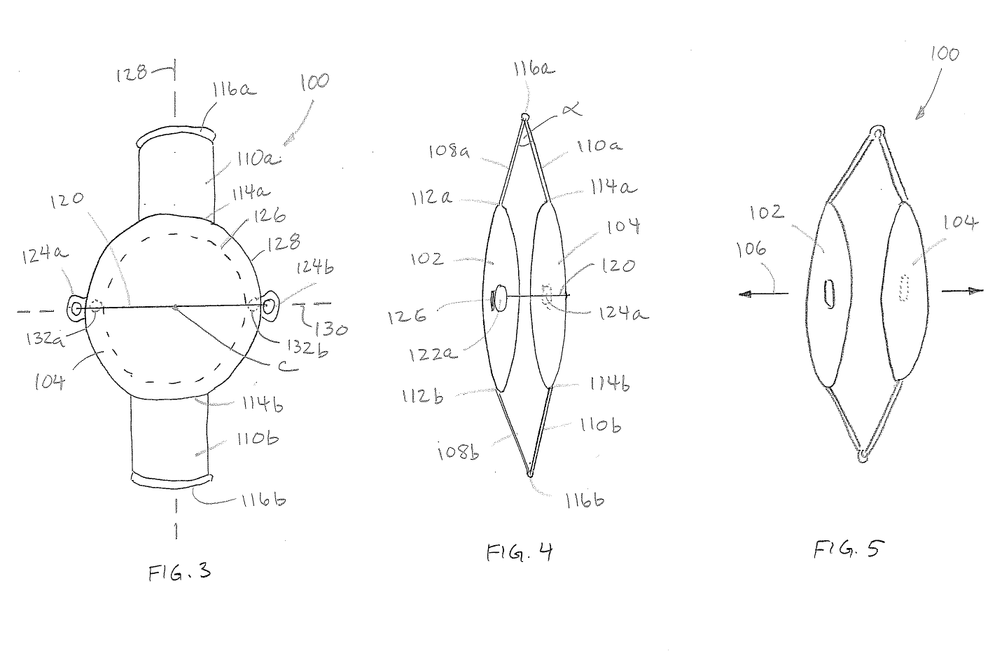

Turning now to FIGS. 3 and 4, an intraocular lens (IOL) 100 according to the invention is shown. The IOL 100 includes a first posterior optic 102 for focusing light and a second anterior optic 104 for focusing light, each aligned and displaceable along a common anterior-posterior axis 106. The first optic 102 has an overall diameter preferably approximately 4 to 7 mm. The second optic 104 has an overall diameter preferably approximately 4 to 7 mm. The optics 102, 104 are manufactured from a fully-polymerized optically transparent material, preferably a silicone. Each optic 102, 104 has a preferably has a different optical power. By way of example, the anterior optic may have a relatively higher and positive optical power, whereas the posterior optic may have a variable and/or negative optical power.

A first set of haptics 108a, 108b is coupled preferably at two diametrically opposed peripheral locations relative to the first optic 102, and a second set of haptics 110a, 110b is coupled preferably at two diametrically opposed peripheral locations relative to the second optic 104. The first set of haptics 108a, 108b joins the first optic 102 at optic haptic junctions 112a, 112b. The second set of haptics 110a, 110b joins the second optic 104 at its own respective optic haptic junctions 114a, 114b. The ends of the first and second sets of haptics preferably join each other at hinges 116a, 116b. The radial edges of the hinges 116a, 116b may optionally include a cuff, fenestrations, or other structure to facilitate adhesion to the capsular bag. In accord with the IOL described herein, the first and second set of haptics 108a, 108b, 110a, 110b are stiffer than the optic-haptic junctions 112a, 112b, 114a, 114b and the hinges 116a, 116b. Also in accord with the described IOL, the IOL is provided with an inherent pre-bias that is adapted to bias the first and second optics 102, 104 away from one another along the anterior-posterior axis 106. Such pre-bias is preferably applied by integration of a bias element at at least one of the haptic-optic junctions 112a, 112b, 114a, 114b, at the hinges 116a, 116b between the first and second sets of haptics 108a, 108b, 110a, 110b, or at a combination thereof. Such bias element may comprise a resilient polymer and may be integrated in the construction of the optic-haptic junction and/or the hinge.

In accord with another aspect of the lens, the IOL 100 is restrained against the pre-bias in a stressed state and within a more planar configuration, with the first optic 102 retained at a first location or within a first distance of the second optic 104. In addition, in the stressed configuration, the angle a between each adjacent haptic (e.g., 108a, 110a coupled to a common hinge 116a) is preferably does not exceed 45° (FIG. 4). A restraining element 120 is provided to the lens 100 for the temporary restraint of the lens in the stressed, planar, non-accommodating configuration during implantation and a post-operative period.

The restraining element 120 comprises an element of material extending across a portion of at least one of the first and second optics 102, 104 within 5 mm of the optical center C, and more preferably within 4 mm (demarcated by boundary line 126) of the optical center of such optic. Importantly, the restraining element 120 extends across the optic and maintains the IOL in the stressed state without reliance on the haptics, and most preferably are rotationally offset from the haptics. The restraining element 120 may be supported by mounts 122a, 122b provided to the periphery 128 of one of the first and second optics, and optionally by mounts 124a, 124b provided to the periphery of the other of the first and second optics. The mounts may include patent holes or other structure to support the restraining element 120. Alternatively, the restraining element may extend through holes or fenestrations 132a, 132b defined directly within (but outside the optically useful portion of) the periphery of the optic. As another option, the first and second optics 102, 104 can be tied together; i.e., trussed without dedicated structure on the lens 400 (either on first or second optics 402, 404 for the restraining element 420 (FIG. 12). The restraining element 120 is of such a length or otherwise dimensioned so as to retain the first and second optics 102, 104 in a defined spaced apart relationship. The restraining element 120 may be a length of suture material with knotted ends 126 coupled to at least mounts 122a, 122b, or within holes 132a, 132b, or extending through such structure and tied or otherwise extending about a portion of the lens 100.

The restraining element 120 preferably extends in a direction transverse to a long axis 128 that is defined across an optic 104 and the set of haptics 110a, 110b extending therefrom. In accord with embodiments, the restraining element 120 extends orthogonal to the long axis 128. In accord with the embodiment shown in FIG. 3, the restraining element 120 extends parallel to a short axis 130 that is orthogonal to the long axis. In accord with such embodiment, the restraining element 120 extends coaxial with the short axis 130. More particularly, the restraining element 120 extends diametrically across the optic 104, and thus directly across and through the optic center C.

The restraining element 120 is preferably constructed of a material that is laser-releasable material, such that the element may be released under the control of an eye surgeon, preferably via a non-surgically invasive means such as via a YAG laser or other applied energy. One preferred material is Ethicon Vicryl® resorbable 9-0 or 10-0 monofilament suture.

Generally, the method for implanting the intraocular lens includes (a) inducing cycloplegia; (b) providing the intraocular lens having a first optic portion, a first set of haptics extending from the first optic portion, a second optic portion, a second set of haptics extending from the second optic portion, a flexible hinge at the ends of the first and second sets of haptics, and an as manufactured inherent bias induced between at least one of (i) the first optic portion and the first set of haptics, (ii) the second optic portion and the second set of haptics, and (iii) and the hinge, the intraocular lens being held in a stressed, planar, non-accommodating state by a restraining element that extends across the lens in a direction that is transverse to the long axis of the intraocular lens such that the intraocular lens has a lower optical power relative to an accommodating non-stressed state of the lens; (c) inserting the stressed state intraocular lens into a capsular bag of the eye; (d) maintaining cycloplegia until the capsular bag physiologically affixes to the intraocular lens; and (e) releasing the restraining means to permit the first and second optic portions of the intraocular lens to move along the anterior-posterior axis 106 relative to each other from the stressed state into the non-stressed state into a configuration in which the intraocular lens has an increased optical power (as shown in FIG. 5), and wherein the optical power of the intraocular lens is reversibly adjustable in response to stresses induced by the eye such that the lens can accommodate. When the restraint is released and the optics can be and are moved away from each other, the angle between the adjacent haptics increases, and the overall diameter of the IOL decreases.

More particularly, according to a preferred method of implantation, the ciliary body muscle is pharmacologically induced into a relaxed stated (cycloplegia), a capsulorrhexis is performed on the lens capsule, and the natural lens is removed from the capsule. The prosthetic lens is then placed within the lens capsule. According to a preferred aspect of the invention, the ciliary body is maintained in the relaxed state for the duration of the time required for the capsule to naturally heal and shrink about the lens; i.e., possibly for several weeks. After healing has occurred, the restraining element 120 is then released under surgeon control to release the lens from the stressed state. The ciliary body and lens may then interact in a manner substantially similar to the physiological interaction between the ciliary body and a healthy natural crystalline lens.

Alternatively, a fully relaxed lens (i.e., without restraining element) can be coupled to a fully stressed and contracted ciliary body.

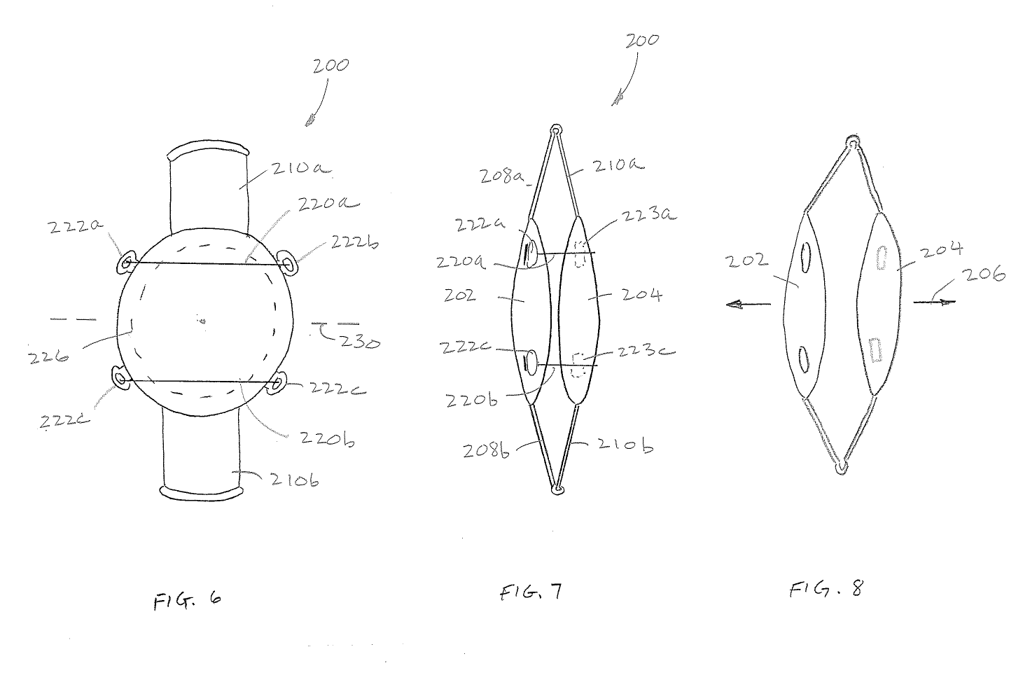

Turning now to FIGS. 6 and 7, another IOL 200 is shown, substantially similar to IOL 100, with differences therefrom now described. The IOL 200 includes a plurality of restraining elements 220a, 220b that extend parallel to each other and the short axis 230, from one of the anterior and posterior optics 202, 204, across portions of the other of the anterior and posterior optics 202, 204, and on opposite sides of the optical center C. Each of the restraining elements 220a, 220b extends within 5 mm of the optical center C (demarcated by an area within boundary 226), and more preferably within 4 mm of the optical center of such optic. Importantly, the restraining elements 220a, 220b, without support of the haptics 208a, 208b, 210a, 210b, extend across the at least one optic. Peripheral mounts 222a, 222b, 223a, 223b (or peripheral holes or fenstrations (shown in FIG. 3)) are provided to support the restraining elements. When both restraining elements 220a, 220b are removed or otherwise released from restraining the optics relative to each other, the first and second optics 202, 204 can move along the anterior-posterior axis 206 relative to each other from the stressed state into the non-stressed state into a configuration in which the intraocular lens has an increased optical power (FIG. 8).

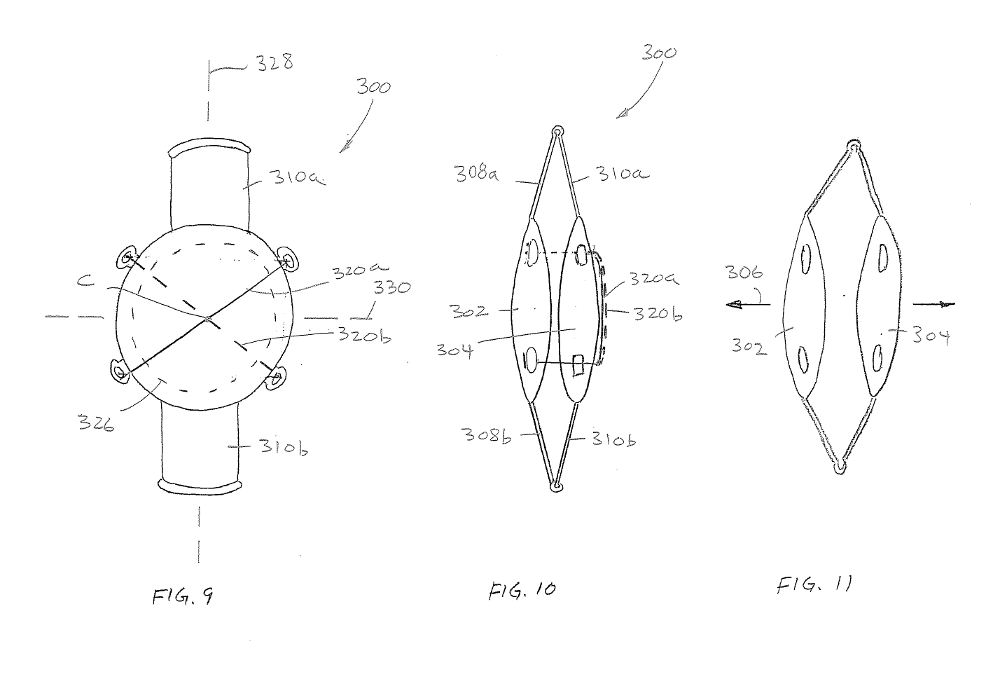

Turning now to FIGS. 9 and 10, another IOL 300 is shown, substantially similar to IOL 200, with differences therefrom now described. The IOL 300 includes at least one restraining element that extends straight across the optic at an angle relative to both the long axis 328 and the short axis 330. More specifically, a plurality of restraining elements 320a, 320b extend at an angle relative to each other, from one of the first and second optics 302, 304, across portions of the other of the first and second optics 302, 304. Each of the restraining elements 320a, 320b extends within 5 mm of the optical center C and more preferably within 4 mm of the optical center of such optic (demarcated by an area within boundary 326), and even more preferably close to or through the optical center C. Importantly, the restraining elements 320a, 320b extend across the at least one optic without support of the haptics 308a, 308b, 310a, 310b. Peripheral mounts 322a, 322b, 323a, 323b (or peripheral holes or fenestrations (shown in FIG. 3)) are provided to support the restraining elements. When both restraining elements 320a, 320b are removed or otherwise released from restraining the optics relative to each other, the first and second optics 302, 304 can move along the anterior-posterior axis 306 relative to each other from the stressed state into the non-stressed state into a configuration in which the intraocular lens has an increased optical power (FIG. 11).

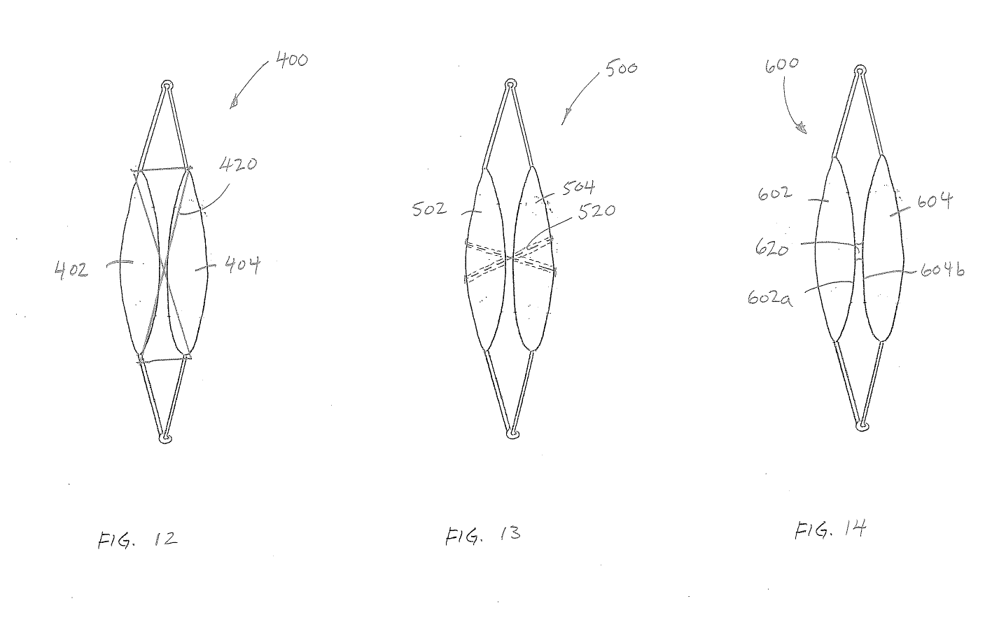

Turning now to FIG. 13, as another option, the first and second optics 502, 504 of the lens 500 can be directly stitched together with the restraining element 520. The stitch or stitches can be at an angle relative to the optical axis (as shown), or extend parallel to the optical axis (anterior-posterior axis), even directly through the optical axis. As yet another option, referring to FIG. 14, the anterior surface 602a of the posterior optic 602 and the posterior surface 604b of the anterior optic 604 (i.e., the two surfaces of the optics which are closest together) can be glued together with a preferably polymeric glue 620. Such glue is adapted to be removable with laser energy to release the IOL from restraint 600, but can be bioresorbable or chemically dissolvable.

The intraocular lens of the invention is compatible with modern cataract surgery techniques. The lens utilizes an axial displacement of one optic relative to another optic to achieve a large increase in optical power of the implanted lens.

There have been described and illustrated herein embodiments of an intraocular lens. While particular embodiments of the invention have been described, it is not intended that the invention be limited thereto, as it is intended that the invention be as broad in scope as the art will allow and that the specification be read likewise. Thus, while two opposing diametric haptics have been described for each optic, it is appreciated that each optic may be provided with more than two haptics; it being appreciated that the restraining element restrains the first and second optics without relying on extension from one haptic, across the optic, and then to another haptic. That is, the restraining element is separate and independent from all structure that mounts the lens to the capsular bag. Also, while the restraining element has been shown as extending across the anterior optic, it may additionally or alternatively extend across the posterior optic. It will therefore be appreciated by those skilled in the art that yet other modifications could be made to the provided invention without deviating from its scope.

What is claimed is:

1. An intraocular lens for replacement of a natural crystalline lens within a capsular bag during eye surgery, said intraocular lens comprising:
a) a first optic adapted to focus light, the first optic having a first periphery;
b) first haptics coupled to the first periphery of the first optic, the first haptics having a first portion joining the first optic at a first optic-haptic junction and having a peripheral second portion;
c) a second optic adapted to focus light, the second optic having a second periphery, wherein an anterior-posterior axis extends through the optical centers of the first and second optics;
d) second haptics coupled to the second periphery of the second optic, the second haptics having a first portion joining the second optic at a second optic-haptic junction and having a peripheral second portion;
e) a hinge structure coupling the second portion of the first haptics and the second portion of the second haptics together such that the first and second haptics are permitted to move relative to each other,
wherein at least one of the first optic-haptic junction, the second optic-haptic junction and the hinge structure is provided with a bias that biases the first and second optics away from each other along the anterior-posterior axis; and
f) a restraining element operating against the bias so as to maintain the first and second optics in a relatively close position and the lens in a stressed state of relatively greater planarity,
the restraining element releasable without an invasive surgical procedure after completion of the eye surgery during which the intraocular lens is implanted, and once the intraocular lens is implanted in the eye and the restraining element is released,
wherein the lens is adapted such that stresses induced by the eye result in the first and second optics moving relative to each other and an optical power of the lens being adjusted in response thereto.

2. An intraocular lens according to claim 1, wherein the first haptics and the second haptics can change angle relative to each other about the hinge structure.

3. An intraocular lens according to claim 1, wherein the first and second haptics are stiffer than the first and second optic-haptic junctions and the hinge structure.

4. An intraocular lens according to claim 1, wherein the restraining element is decoupled from the first haptics and the second haptics.

5. An intraocular lens according to claim 1, wherein the restraining element is decoupled from all structure that mounts the lens to the capsular bag.

6. An intraocular lens according to claim 1, wherein the restraining element extends within a boundary that is within 5 mm of an optical center of one of the first and second optics.

7. An intraocular lens according to claim 1, wherein the restraining element extends within a boundary that is within 4 mm of an optical center of one of the first and second optics.

8. An intraocular lens according to claim 1, wherein the restraining element extends diametrically across one of the first and second optics, without support of the haptics.

9. An intraocular lens according to claim 1, wherein each of the first and second optics has a long axis defined across the respective optic and the respective haptics extending therefrom, and the restraining element extends in a direction transverse to the long axis.

10. An intraocular lens according to claim 9, wherein the restraining element extends orthogonal to the long axis.

11. An intraocular lens according to claim 9, wherein the restraining element extends parallel to a short axis orthogonal to the long axis.

12. An intraocular lens according to claim 11, wherein the restraining element extends coaxial with the short axis.

13. An intraocular lens according to claim 9, wherein the restraining element extends at an angle relative to each of the long and short axes.

14. An intraocular lens according to claim 1, wherein at least one of the first and second optics includes at least one peripheral mount rotationally offset from the first haptics and second haptics and to which the restraining element is coupled.

15. An intraocular lens according to claim 1, wherein at least one of the first and second optics includes peripheral fenestrations through which the restraining element is received.

16. An intraocular lens for replacement of a natural crystalline lens within a capsular bag during eye surgery, said intraocular lens comprising:
a) a first optic adapted to focus light at a first optical power, the first optic having a first periphery;
b) first haptics coupled to the first periphery of the first optic, the first haptics having a first portion joining the first optic at a first optic-haptic junction and having a peripheral second portion;
c) a second of a optic adapted to focus light at a second optical power different from the first optical power, the second optic having a second periphery, wherein an anterior-posterior axis extends through the optical centers of the first and second optics;
d) second haptics coupled to the second periphery of the second optic, the second haptics having a first portion joining the second optic at a second optic-haptic junction and having a peripheral second portion;
e) a hinge structure coupling the second portions of the first haptics and the second haptics together such that the first and second haptics are permitted to move relative to each other, the first and second haptics being stiffer than the hinge structure,
wherein at least one of the first optic-haptic junction, the second optic-haptic junction and the hinge structure is provided with a bias that biases the first and second optics away from each other along the anterior-posterior axis; and
f) a restraining element operating against the bias so as to maintain the first and second optics in a relatively close position and the lens in a stressed state at a first diameter and with adjacent ones of the first and second haptics joined at the hinge structure held a first angle,
the restraining element releasable without an invasive surgical procedure after completion of the eye surgery during which the intraocular lens is implanted, and once the intraocular lens is implanted in the eye and released into a non-stressed state, the lens is adapted to assume a smaller second diameter and an increase in the angle between the adjacent ones of the first and second haptics joined at the hinge structure,
wherein the lens is adapted such that stresses induced by the eye result in the first and second optics moving relative to each other and an optical power of the lens being adjusted in response thereto.

17. An intraocular lens according to claim 16, wherein the restraining element extends from one of the first and second optics to the other of the first and second optics.

18. An intraocular lens according to claim 16, wherein the restraining element extends from a periphery of one of the first and second optics to the other of the first and second optics.

19. An intraocular lens according to claim 16, wherein the restraining element extends as a stitch from one of the first and second optics to the other of the first and second optics.

20. An intraocular lens according to claim 16, wherein the restraining element is a releasable glue that couples the first and second optics together.

21. An intraocular lens according to claim 16, wherein the angle in the stressed state does not exceed 45°.

22. An intraocular lens for replacement of a natural crystalline lens within a capsular bag during eye surgery, said intraocular lens comprising:
a) a first optic adapted to focus light at a first optical power;
b) a second optic adapted to focus light at a second optical power, the first and second optics connected by haptics, wherein a bias is provided that biases the first and second optics away from each other to bias the lens into a configuration in which the lens assumes a first diameter; and
c) a temporary restraining element that operates against the bias so as to maintain the first and second optics in a first proximity and the lens in a stressed state and at a relatively larger second diameter,
the restraining element releasable without an invasive surgical procedure after completion of the eye surgery during which the intraocular lens is implanted, and once the intraocular lens is implanted in the eye and released into a non-stressed state, the lens is adapted to reconfigure from having the second diameter to having the first diameter in response to stresses induced by the eye, and an optical power of the lens being adjusted in response to such reconfiguration.

23. An intraocular lens according to claim 22, wherein the restraining element is decoupled from the haptics.
